(19) United States
(12) Patent Application Publication
(10) Pub. No.: US 2003/0064081 A1
(43) Pub. Date: Apr. 3, 2003

(54) Topical anti-pruritic composition

(76) Inventor: William H. Morgan, Delton, MI (US)

Correspondence Address: PRICE HENEVELD COOPER DEWITT & LITTON, 695 KENMOOR, S.E., P O BOX 2567, GRAND RAPIDS, MI, 49501, US

(21) Appl. No.: 10/227,894

(22) Filed: Aug. 26, 2002

Related U.S. Application Data
(60) Provisional application No. 60/326,273, filed on Oct. 1, 2001.

(51) Int. Cl. A61K 31/192; A61K 9/00

(52) U.S. Cl. 424/400; 514/570

(57) ABSTRACT

A topical antiallergenic composition and methods for treatment are provided. A proprionic acid nonsteroidal anti-inflammatory drug is prepared in a suitable topical form for the treatment of skin conditions caused by allergic reactions. The various allergic reactions include poison ivy, poison oak, poison sumac, mosquito bites, eczema and other allergic skin reactions.

### CROSS-REFERENCE TO RELATED APPLICATION

This application claims the benefit of U.S. Provisional Application No. 60/326,273, entitled TOPICAL ANTI-PRURITIC COMPOSITION, filed Oct. 1, 2001.

### BACKGROUND OF THE INVENTION

The present invention relates to the treatment of skin conditions caused by exposure to allergens resulting in allergic reactions. Such allergen sources and their subsequent skin reactions include poison ivy, poison oak, poison sumac, mosquito bites, eczema and other allergic skin reactions.

Exposure to such allergens results in some of the most common topical allergic reactions in the United States. There are a variety of conventional products used to treat poison ivy, poison oak, poison sumac, mosquito bites, eczema and other allergic skin reactions. Typical products include oral antihistamines, such as Claritin® or Diphenhydramine, and/or a corticosteroid with anti-inflammatory properties, such as Prednisone. There are several topical products also available including topical antihistamine products, such as Diphenhydramine containing medicaments, and topical steroid products, such as Betamethasone or Hydrocortisone containing medicaments. Additional products used in topical treatment include antipruritic products, such as Calamine containing medicaments, cleansing wipes for removal of an allergen, or topical astringent solutions such as Domeboro®.

### SUMMARY OF THE INVENTION

The present invention comprises a topical antiallergenic composition comprising as its principle ingredient a proprionic acid nonsteroidal anti-inflammatory drug. It is surprising to find that proprionic acid nonsteroidal anti-inflammatory drugs show topical antiallergenic properties.

These and other features, advantages, and objects of the present invention will be further understood and appreciated by those skilled in the art by reference to the following specification and claims.

### DETAILED DESCRIPTION OF PREFERRED EMBODIMENT

In the product of the preferred embodiment, a proprionic acid nonsteroidal anti-inflammatory drug is prepared in a suitable topical form. The various topical forms include a paste, cream, gel or liquid. Medications included in the class of proprionic acid nonsteroidal anti-inflammatory drugs are fenoprofen, fluribuprofen, ibuprofen, ketoprofen and naproxen, and their corresponding pharmaceutically acceptable salts.

The dosage of active per gram of paste, cream, gel or liquid is approximately the same as the active dosage per gram in the tablet form of the same active. Thus, the ibuprofen topical dosage range is from about 150 mg to about 850 mg per gram of product, the fenoprofen topical dosage range is from about 550 mg to about 650 mg per gram, the fluribuprofen topical range is from about 10 mg to about 150 mg per gram, the ketoprofen topical dosage range is from about 10 mg to about 75 mg per gram and the naproxen topical dosage range is from about 170 to about 600 mg per gram.

The most preferred proprionic acid nonsteroidal anti-inflammatory drug known at present time is ibuprofen. The most preferred form known at the present time is a cream or paste.

The cream or paste can be made for example by adding water to the conventional proprionic acid nonsteroidal anti-inflammatory tablets. More specifically, the cream or paste can be made by combining 6 drops of hot tap water (100-150° F.) with three 200 mg ibuprofen tablets. From the onset of said combination to approximately 15 minutes thereafter, stirring occasionally, the hot water will cause the dissolution of the ibuprofen tablets. A thick pourable consistency will result with an approximate tablet weight to water volume ratio of 10000:1. Conceivably, one may also use ibuprofen powder as a source of the proprionic acid nonsteroidal anti-inflammatory drug.

Ingredients in a paste or cream made directly from the tablets include excipients such as Carnauba wax, corn starch, hydroxypropyl methylcellulose, propylene glycol, silicon dioxide, pregelatinized starch, stearic acid, titanium dioxide, iron oxide black, as well as the proprionic acid nonsteroidal anti-inflammatory drug as the active ingredient.

In the above formula, water is the carrier while the tablet polymetric coating material makes the paste more cream like. Other pharmaceutical carriers that might be used include petrolatum, lanolin, Aquaphor®, Polysorb, Nivea® cream and polyethylene glycol.

The product of the preferred embodiment is effective against itching resulting from poison ivy, poison oak, poison sumac, mosquito bites, eczema and other allergic skin reactions. Upon topical application of the product of the preferred embodiment, dissipation of the underlying source of itch can be expected within two to four days. Generally, a person can expect relief of itch symptoms within 5 to 15 minutes of topical application.

The above description is considered that of the preferred embodiments only. Modifications of the invention will occur to those skilled in the art and to those who make or use the invention. Therefore, it is understood that the embodiments described above is/are merely for illustrative purposes and not intended to limit the scope of the invention, which is defined by the following claims as interpreted according to the principles of patent law, including the Doctrine of Equivalents.

The invention claimed is:

1- A topical antiallergenic composition comprising as its principle ingredient a proprionic acid nonsteroidal anti-inflammatory drug.

2- The topical antiallergenic composition of claim 1, wherein said proprionic acid nonsteroidal anti-inflammatory drug is ibuprofen.

3- The topical antiallergenic composition of claim 1, wherein said composition is in a paste form.

4- The topical antiallergenic composition of claim 1, wherein said composition is in a cream form.

5- The topical antiallergenic composition of claim 1, wherein said composition is in a gel form.

6- The topical antiallergenic composition of claim 1, wherein said preferred composition is in a liquid form.

7- A process for producing the topical antiallergenic composition of claim 1, wherein water is heated to a temperature of 100 to 150 degrees Fahrenheit; the heated water is then added to said proprionic acid non-steroidal anti-inflammatory drug ibuprofen.

8- A method for treating allergic skin reactions comprising:
applying to the affected area of the skin a topical antiallergenic composition containing as its principle ingredient a proprionic acid nonsteroidal anti-inflammatory drug.

9- The method of claim 8, wherein the allergic skin reaction is poison ivy.

10- The method of claim 8, wherein the allergic skin reaction is poison oak.

11- The method of claim 8, wherein the allergic skin reaction is poison sumac.

12- The method of claim 8, wherein the allergic skin reaction is caused by mosquito bites.

13- The method of claim 8, wherein the allergic skin reaction is eczema.

14- The method of claim 8, wherein said proprionic acid nonsteroidal anti-inflammatory drug is ibuprofen.

15- The method of claim 8, wherein said composition is in a paste form.

16- The method of claim 8, wherein said composition is in a cream form.

17- The method of claim 8, wherein said composition is in a gel form.

18- The method of claim 8, wherein said preferred composition is in a liquid form.
